(19) United States
(12) Patent Application Publication
(10) Pub. No.: US 2006/0025624 A1
(43) Pub. Date: Feb. 2, 2006

(54) Method for the catalyst-free production of alkoxybenzonitriles

(75) Inventors: Mario Gomez Andreu, Wasserburg (DE); Jurgen Sans, Trostberg (DE); Franz Thalhammer, Trostberg (DE)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(73) Assignee: Degussa AG, Trostberg (DE)

(21) Appl. No.: 10/515,090

(22) PCT Filed: May 16, 2003
(86) PCT No.: PCT/EP03/05176

§ 371 Date: Jun. 30, 2005

(30) Foreign Application Priority Data
May 17, 2002 (DE) 102 22 330.0

(51) Int. Cl. C07C 253/30

(52) U.S. Cl. 558/423

(57) ABSTRACT

A process is claimed for the catalyst-free production of alkoxybenzonitriles by substituting monohalogenated starting compounds of the general formula I 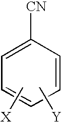
in which X denotes Cl or Br and Y denotes H, alkyl or aryl, characterized in that the starting compound and an alcoholate of the general formula Z-OAlk in which Z=Na⁺ or K⁺ and Alk=C₁ to C₄ alkyl are held for 2 to 10 hours at the final temperature after heating both to 90 to 250° C., then the reaction mixture is cooled to room temperature, is restirred after adding water and finally the end product is separated. Using this process in which preferably benzonitriles chlorinated and/or brominated in the 2- or 4-position are used as the starting compound and sodium methylate or sodium ethylate is used as the alcoholate, it is possible to economically produce the respective end products in very good yields and with a high purity in an efficient and at the same time environmentally friendly manner.

The present invention concerns a process for the catalyst-free production of alkoxybenzonitriles by substituting monohalogenated starting compounds of a defined formula.

Alkoxybenzonitriles are very important intermediates in the synthesis of numerous agricultural and pharmaceutical products.

Although corresponding production processes are well known from the general literature, most of these processes use special catalysts.

In the catalyst-free processes known in the literature, the starting compounds are not aromatic nitrites.

In general the known processes can be compiled into the following groups:
Substitution of halogens in the form of bromine or chlorine by a methylate on phenyl rings which carry no nitrile group and which are not aromatic nitrites. Copper or palladium catalysts are used for this purpose. According to U.S. Pat. No. 5,965,766, U.S. Pat. No. 5,917,079 and EP-PS 831 083 alkoxybenzene derivatives are prepared by reacting aromatic halogenides with metal alcoholates. Only the reaction of these intermediate products with copper or potassium cyanide in the presence of a catalyst leads to alkoxybenzonitriles.
Substitution of halogens in the form of bromine or chlorine by a methylate on phenyl rings which carry at least one nitro group and which are likewise not aromatic nitrites. In this case a catalyst is not usually used since nitrobenzenes are known to be more active substrates than for example benzonitriles.

Examples of this are the patent documents JP 11 147 859, JP 07 316 102, EP 496 370 and DE 35 26 930.
Processes in which transition metal catalyst are used: According to J. Am. Chem. Soc., 1997, 119, 3395-3396; J. Org. Chem. 1997, 62, 5413-5418 and WO 98/15515 halogenated benzonitriles are reacted with metal alcoholates using an expensive palladium or nickel compound in combination with correspondingly expensive ligand as a catalyst. It is also known that halogenated aryls can be catalytically dehalogented with the aid of a Pd/imidazole salt system according to Organometalics, 2001, 20, 6, 3607-3612.
Processes in which highly activated halobenzonitriles (polyhalogenated benzonitriles or halogenated polynitrilebenzene derivatives) are directly reacted with metal alcoholates. According to JP 50121424 1,3-dicyano-2,4,5,6-tetrachloroisophthalodinitrile is reacted with sodium methylate to form 1,3-dicyano-4-methoxy-2,5,6-trichloroisophthalodinitrile; CS 256818 and CS 261149 recommend the direct reaction of 2,6-dichlorobenzonitrile with 4 equivalents of sodium methylate at boiling temperature to form 2-chloro-6-methoxybenzonitrile in which a yield between 75 and 80% is achieved. However, this variant is limited to highly activated nitrites.

Processes for producing alkoxylated benzonitriles from non-activated benzonitriles which do not require participation of transition metal catalysts are not known in the prior art.

Hence on the basis of the described disadvantages of the prior art the object of the present invention is to provide a process for the catalyst-free production of alkoxybenzonitriles. The target compounds should be obtained in this process as directly as possible, in good yields and purities and the process should completely dispense with the use of expensive and environmentally questionable substances.

This object was achieved by a corresponding process in which monohalogenated benzonitriles of the general formula I 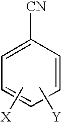
in which X denotes Cl or Br and Y denotes H, alkyl or aryl are reacted in a substitution reaction wherein the starting compound and an alcoholate of the general formula Z-OAlk in which Z=Na⁺ or K⁺ and Alk=C₁ to C₄ alkyl, are held for 2 to 10 hours at the final temperature after both are heated to 90 to 250° C. The process according to the invention additionally preferably comprises one or more steps selected from cooling the reaction mixture to 10 to 40° C., in particular to room temperature e.g. 18 to 27° C., adding water to the reaction mixture or/and separating the final product. After holding the reaction mixture at the final temperature, the reaction mixture is particularly preferably cooled to 10 to 40° C. in particular to room temperature, restirred after adding water and finally the end product is separated.

The residue Y can be located in the ortho, meta or para position relative to the CN group. If Y represents an alkyl group, it is preferably a C₁-C₃₀, in particular a C₁-C₆ and most preferably a C₁-C₄ alkyl group. The alkyl group can be straight chained or branched. If Y denotes aryl, it is preferably a C₄-C₃₀, more preferably a C₅-C₂₀ and most preferably a C₆-C₁₅ aryl residue.

Surprisingly in connection with this process, it has turned out that it is not only possible to produce the respective end products without participation of the previously used catalysts that have been regarded as being necessary, but that this is also possible in yields of usually above 90%. Furthermore, the time required up to the end of reaction is within economic limits and thus the process according to the invention also has considerable advantages from an economic point of view. In this connection the fact that the new process can also be carried out in a very environmentally friendly and economical manner since it does not require catalysts and only alkali salts are formed as by-products, is of primary importance. This was unexpected.

According to the present invention benzonitriles that are chlorinated or brominated in the 2- or 4-position whose production is well-known from the literature are preferably used as monohalogenated benzonitriles.

Sodium methylate or sodium ethylate are regarded as preferred alcoholates.

The reaction is carried out at temperatures between 90 and 250° C., but preferably temperatures have proven to be particularly suitable which are between 100 and 150° C. and particularly preferably between 115 and 140° C.

After the respective temperature upper limit has been reached, the reaction mixture should be held according to the invention for a period of 2 to 10 hours and in particular for 3 to 5 hours at the respective final temperature. Under these conditions of reaction engineering the pressure of course increases in a closed system such as an autoclave in which case pressure ranges of usually 2 to 20 bar are acceptable and a pressure of ca. 3 to 5 bar is recommended.

In order to avoid an excessive increase in pressure due to uncontrolled release of reaction heat or to reduce the pressure increase, the reaction mixture can also be held for a relatively long period of for example 3 hours under reflux and only subsequently be reacted in an autoclave for a further 2 to 3 hours at the final temperature.

If the reaction is not carried out in a closed system such as an autoclave, it is also possible to carry out the process completely under reflux which, however, increases the reaction times.

In order to exclude possible side reactions and for reasons of safety, the present invention preferably provides that the reaction is carried out under inert gas and in particular under nitrogen atmosphere.

The amount of alcoholate used in the claimed process is not critical and can be between 70 mol % and 400 mol % based on the amount of monohalogenated benzonitrile that is used. However, it is recommended to use an amount of alcoholate between 100 and 200 mol % based on the halogenated benzonitrile that is used and especially between 105 and 115 mol %.

Within the scope of the invention it is also regarded as preferable to use the metal alcoholate in the form of a corresponding alcoholic solution. The concentration of this solution is also uncritical and can be between 15 and 50% by weight. However; it is recommended that the concentration of the alcoholate solution is between 20 and 40% by weight and in particular between 25 and 35% by weight.

The subsequent separation of the product is carried out by standard methods, where of course filtration is recommended for solid products and phase separation is recommended for liquid products. Finally the residual moisture can be removed from the product if required by mild temperatures of ca. 30 to 50° C.

At the end of the reaction the reaction mixture is usually a suspension of a sodium and/or potassium salt and proportions of alkoxybenzonitrile in the corresponding alcohol. The product can be readily separated from this system by adding water and restirring. In this process the salt is completely dissolved and in the case of solid benzonitrile derivatives the product precipitates. It is particularly advantageous to cool the resulting suspension while mixing thoroughly, in the course of which the product is obtained practically quantitatively. The crystals that are obtained in this manner can be separated from the liquid phase in any conventional manner. The product is usually dried at pressures between 0.1 to 1000 mbar and at temperatures between 20 and 40° C., pressures between 10 and 20 mbar and temperatures between 25 and 35° C. being particularly recommended.

In the case of benzonitrile derivatives that are liquid at room temperature, two phases form after adding water which are separated; then the product is purified by distillation. The subsequent drying can be carried out as already described.

Overall the process according to the invention is characterized by being technically very simple to carry out and also by its environmental friendliness. Only sodium or potassium halogenides are formed as by-products of the reaction which additionally underlines the environmental friendliness and efficiency of the proposed process.

Overall it may be stated that by using the catalyst-free process for producing alkoxy-benzonitriles according to the invention, the respective products are obtained in an economically and ecologically considerably improved manner, in high purity and in large yields.

The following examples illustrate the advantages associated with the process according to the invention.

### EXAMPLES

### Example 1

41.3 g 4-chlorobenzonitrile and 63 g of a 30% solution of sodium methylate in methanol are placed in an autoclave while stirring. The reactor is closed and the reaction mixture is heated to 120° C. and stirred at this temperature for 3 hours. The reactor is allowed to cool. 100 ml water is added and restirred for about 15 minutes at room temperature. The suspension that forms is suction filtered, the product is washed with water and subsequently dried in a drying cabinet at 40° C. Yield: 37.9 g 4-methoxybenzonitrile (100% GC, 95% of theory).

### Example 2

41.3 g 4-chlorobenzonitrile and 63 g of a 30% solution of sodium methylate in methanol are placed in an autoclave while stirring. The reaction mixture is heated to boiling temperature and stirred at this temperature for 3 hours under reflux. Subsequently the reactor is closed and it is restirred at 120° C. for about 1 hour. The reactor is allowed to cool. 100 ml water is added and restirred for about 15 minutes at room temperature. The suspension that forms is suction filtered, the product is washed with water and subsequently dried in a drying cabinet at 40° C. Yield: 36 g 4-methoxybenzonitrile (100% GC, 90% of theory).

1. Process for the catalyst-free production of alkoxybenzonitriles by substituting a monohalogenated starting compound of the general formula I 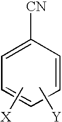
in which X denotes Cl or Br and Y denotes H, alkyl or aryl, characterized in that the starting compound and an alcoholate of the general formula Z-OAlk in which Z=Na⁺ or K⁺ and Alk=C₁ to C₄ alkyl, the alcoholate being used in the form of an alcoholic solution of the respective alcoholate, are held for 2 to 10 hours at the final temperature after heating both to 90 to 250° C. to form a reaction mixture.

2. Process as claimed in claim 1, additionally comprising the step of cooling the reaction mixture to 10° C. to 40° C., in particular to room temperature.

3. Process as claimed in claim 1, additionally comprising the step of adding water to the reaction mixture.

4. Process as claimed in claim 1, additionally comprising the step of separating the end product of alkoxybenzonitriles from the reaction mixture.

5. Process as claimed in claim 1, characterized in that benzonitriles chlorinated or brominated in the 2- or 4-position are used as the starting compound.

6. Process as claimed in claim 1, characterized in that sodium methylate or sodium ethylate is used as the alcoholate.

7. Process as claimed in claim 1, characterized in that the alcoholate component is added in amounts between 70 and 400 mol %, based on the halogenated benzonitrile that is used, in particular in amounts between 100 and 200 mol % and especially preferably between 105 and 115 mol %.

8. Process as claimed in claim 1, characterized in that the concentration of the alcoholic solution of the respective alcoholate is preferably 15 to 50% by weight, preferably 20 to 40% by weight and particularly preferably 25 to 35% by weight.

9. Process as claimed in claim 1, characterized in that the reaction mixture is heated to 100 to 150° C. and particularly preferably to 115 to 130° C.

10. Process as claimed in claim 1, characterized in that the reaction mixture is held for 3 to 5 hours at the reaction temperature.

11. Process as claimed in claim 10, characterized in that the reaction is carried out under reflux.

12. Process as claimed in claim 1, characterized in that the reaction is carried out under inert gas and in particular under nitrogen atmosphere.

13. Process as claimed in claim 1, wherein the reaction mixture comprises the starting compound and the alcoholate.

14. Process as claimed in claim 13, wherein the reaction mixture further comprises alkoxybenzonitriles.

15. Alkoxybenzonitrile made according to the process of claim 1.
